(19) United States
(12) Patent Application Publication
(10) Pub. No.: US 2010/0055165 A1
(43) Pub. Date: Mar. 4, 2010

(54) LIPOSOMAL GEL PHTHALOCYANINE PREPARATION FOR PHOTODYNAMIC THERAPY OF TUMORS AND ITS MANUFACTURING

(76) Inventors: Petr Jezek, Praha (CZ); Milos Nekvasil, Praha (CZ); Pavla Pouckova, Dobrichovice (CZ)

Correspondence Address: NOTARO & MICHALOS P.C., 100 DUTCH HILL ROAD, SUITE 110, ORANGEBURG, NY, 10962-2100, US

(21) Appl. No.: 12/447,750

(22) PCT Filed: Nov. 27, 2007
(86) PCT No.: PCT/CZ07/00107

§ 371 Date: Apr. 29, 2009

(30) Foreign Application Priority Data
Nov. 28, 2006 (CZ) PV 2006-743

(51) Int. Cl. A61K 9/127; A61K 31/407; A61P 35/00

(52) U.S. Cl. 424/450; 514/410

(57) ABSTRACT

Liposomal gel hydrophobic phthalocyanine (FCH) preparation for photodynamic therapy of tumors and other diseases is composed of lecithin liposomes or liposomes on the basis of other lipids, with incorporated curing drug, which can be chosen either from a group including hydrophobic hydroxyaluminum phthalocyanine, hydrophobic aluminum phthalocyanine, hydrophobic zinc phthalocyanine, hydrophobic silicone phthalocyanine, or organic silicone phthalocyanine, or hydrophobic phthalocyanine without the core metal; while resulting liposomes are mixed in ratios of 10:1 to 1:10 with a translucent gel, advantageously on the basis of carboxymethylcellulose. The added curing drug can be coated by glucose or other saccharides, by polyethylenglycol or other usable polymers, as lecithin or other lipids, or by sodium chloride or other salts usable in pharmacology. Liposomal gel hydrophobic phthalocyanine (FCH) preparation is manufactured on the following schema: Lecithin or other lipid of a pharmacological purity at concentration between 10 to 40 mg per ml of sterile isotonic solution is fluidised on a microfluidizer in particular chamber to the final particle size smaller than 1000 nm, in temperature higher than 0° C. and a pressure at 500 to 2000 Bar; then while stirring the curing drug or the treated curing drug is added in the ratio of 5:1 to 0.1:1 in relation to the lecithin or other lipid; the resulting suspension is again fludized on a microfludizer in particular smaller chamber to the final particle size smaller than 500 nanometer, with pressure at least 1000 to 2000 Bar, in temperature higher than 0° C.; the resulting suspension is then mixed with a translucent gel in ratios of 10:1 to 1:10; Alternatively, first lecithin or other lipid of a pharmacological purity at concentration between 10 to 40 mg per ml of sterile isotonic solution is microfludized on a microfluidizer in particular chamber to the final particle size smaller than 1000 nm, a pressure at least 1000 to 2000 Bar and temperature higher than 0° C.; Afterwards, in parallel the curing drug or the treated curing drug is separately microfluidized in amounts corresponding to the ratio of 5:1 to 0.1:1 in relation to the lecithin or other lipid in an equal volume of fluid, advantageously on the basis of sterile isotonic solution to the final particle size smaller than 1000 nanometer, and with pressure at 1000 to 2000 Bar; Afterwards both microfludized components are mixed together and again microfludized on a microfludizer in particular chamber with a pressure at 1000 to 2000 Bar and temperature higher than 0° C. to the final particle size maximum of 500 nanometer, the resulting suspension is fluidised on a microfluidizer in a particular smaller chamber with a pressure at least 1000 to 2000 Bar and temperature higher than 0° C. to the final particle size smaller then 500 nm, the resulting suspension is then mixed with a translucent gel in ratios of 10:1 to 1:10; Alternatively, Lecithin or other lipid in the pharmacological purity at the concentration of 10 to 40 mg per milliliter of sterile isotonic solution is first treated by extrusion across the filters with a size 10 to 500 nm together with the curing drug or the treated curing drug in the ratio of 5:1 to 0.1:1 related to the lecithin or other lipid; and the resulting suspension is again fluidised on a microfluidizer in a particular chamber with a pressure at least 1000 to 2000 Bar and temperature higher than 0° C. to the final particle size smaller then 500 nm, with a pressure at least 1000 to 2000 Bar and temperature higher than 0° C., the resulting suspension is mixed with a translucent pharmaceutical gel in a ratio of 10:1 to 1:10.

The liposomal gel hydrophobic phthalocyanine (FCH) preparation for photodynamic therapy of tumors and other diseases: In therapeutic use the preparation is applied onto the tumor surface or the pathological parts of the body, the preparation is let to act for the time period of one minute up to 24 hours, and afterwards, the location is irradiated or illuminated by the coherent or noncoherent light of the wavelength between 500 to 800 nm and intensity of at least 1 J/cm². The resulting gel containing liposomes with incorporated curing drug is during the therapy applied onto surface tumors in dermatology or accessible tumors for light-delivering endoscopes and ideally after several minutes is irradiated or illuminated by the coherent or noncoherent light of the desired wavelength. The suggested system enables instant penetration of the curing drug into the tumor and nearly instant irradiation or illumination (in a minute time intervals from the application) with subsequent curing effect on the tumor. Such a high disintegration effect of the suggested therapy is determined by the suggested composition of the gel.

### TECHNICAL FIELDS

The technical solution concerns an application gel with liposomes for photodynamic therapy of tumor diseases, which contains hydrophobic form of hydroxyl-aluminum phthalocyanine (or aluminum substituted by Si, Zn, and other metals or without a metal core) (referred further to as FCH) modified for subsequent treatment by a microfludizer. The resulting gel containing liposomes with the incorporated curing drug is applied as a therapy on surface tumors in dermatology or on other tumors accessible to light or lightguides, and after several minutes is illuminated by light of desired wavelength. The suggested system allows instant penetration of the curing drug into the tumor and nearly instant illumination (in minute intervals from the application) with following disintegration effect on the tumor. This highly efficient disintegration result of the therapy is determined by the suggested system.

### BACKGROUND ART

Photodynamic therapy, used in a cure of surface tumors, especially in dermatology, lies in a procedure, that the curing drug is incorporated in a gel, which is applied onto a tumor and after a sufficient time illuminated by the light of the desired wavelength.

For photodynamic therapy of tumor diseases were developed following hydrophobic preparations clinically tested (some only pre-clinically) for intervals between application and illumination (T_{DL}) in a range from one hour and longer (up to several days, see data in parentheses): benzyl ester of delta aminolevulic acid, Benzvix (T_{DL} 4 to 6 hours) registered in EU for therapy of gastrointestinal tumors, U.S. Pat. No. 6,492,420, (comp. Photocure, Oslo, Norway); Temoporfin or Foscan (methyl-tetrahydroxyphenyl chlorine, (T_{DL} 96 hours), WO 0166550, Biolitec Pharma, Scotland, United Kingdom) approved in EU for palliative head and neck tumors, prostatic tumors and pancreatic tumors; Benzoporphyrine derivative, alias Verteporfin (BPD-MA, Visudyne, Novartis, UK), which is in phase III of clinical testing for skin amelanotic melanomas; and silicone phthalocyanine also in phase III of clinical testing for curing of skin tumors including Bowen diseases and actinic keratosis, so far with shortest T_{DL} 1 hour.

The suggested solution of a system of hydrophobic phthalocyanine incorporated in liposomes by microfludization and applied in a translucent gel exhibited T_{DL} of 5 to 15 minutes in preclinical testing.

### DISCLOSURE OF THE INVENTION

Liposomal gel hydrophobic phthalocyanine (FCH) preparation for photodynamic therapy of tumor diseases is composed by a system of lecithin liposomes or liposomes on the basis of other lipids, with incorporated curing drug, chosen from a group including hydrophobic hydroxyaluminum phthalocyanine, hydrophobic aluminum phthalocyanine, hydrophobic zinc phthalocyanine, hydrophobic silicone phthalocyanine or organic silicone phthalocyanine, or hydrophobic phthalocyanine without the core metal, mixed in ratios of 10:1 to 1:10 with a translucent gel, advantageously on the basis of carboxymethylcellulose. The incorporated curing drug can be coated by glucose or other saccharides, by polyethyleneglycole, by lecithin or other lipids, or by sodium chloride or other salts sufficient in pharmacology. The Liposomal gel hydrophobic phthalocyanine (FCH) preparation is manufactured so that the lecithin or other pharmaceutical pure lipid in a concentration of 1 to 40 mg per milliliter of fluid, advantageously fluid as a sterile isotonic solution, is microfluidized on a microfludizer in a proper chamber until the final particle size smaller than 1000 nanometers, at the temperature higher than 0 ° C. and a pressure of 500-2000 Bar; afterwards while constantly stirring the curing drug is added or the treated curing drug according to the points 2 to 5 at ratios between 5:1 to 0.1:1 in relation to the lecithin (lipid); the resulting suspension is again microfluidized on a microfluidizer in proper smaller chamber until the final particle size smaller than 500 nm, a pressure of 1000-2000 Bar, and at the temperature higher than 0° C.; the resulting suspension is afterwards mixed with a translucent pharmaceutical gel in ratios between 10:1 to 1:10; alternatively lecithin or other lipid of a pharmaceutical purity in concentration of 1 to 40 mg per ml of fluid, advantageously fluid as a sterile isotonic solution, is microfludized on a microfludizer in a proper chamber until the final particle size smaller than 1000 nm, a pressure minimum of 1000-2000 Bar, at the temperature higher than 0° C.; afterwards the curing drug or the treated curing drug according to the points 2 to 5 of the claims is solitary microfludized on a microfludizer in a desired chamber in amounts corresponding to 5:1 to 0.1:1 ratios related to the lecithin (lipid) in an equal volume of fluid, advantageously of isotonic solution to the final particle size smaller than 1000 nm and a pressure minimum of 1000-2000 Bar, afterwards both microfludized components are mixed together and again microfludized on a microfludizer in the proper smaller chamber with a pressure minimum of 1000-2000 Bar, at the temperature higher than 0° C. until the final particle size maximum of 500 nm; the resulting suspension is microfludized on a microfludizer in the proper smaller chamber with a pressure minimum of 1000-2000 Bar, at the temperature higher than 0° C. until the final particle size smaller than 500 nm; the resulting fluid is then mixed with a translucent pharmaceutical gel in ratios of 10:1 to 1:10; or, alternatively, lecithin or other lipid in a pharmaceutical purity at the concentration of 1 to 40 mg per milliliter of fluid, advantageously fluid as a sterile isotonic solution, is treated by extrusion through the filters of sizes 10 to 500 nm together with a curing drug or the treated curing drug according to the points 2 to 5 of the claims in ratios of 5:1 to 0.1:1 related to the lecithin (lipid); the resulting suspension is further microfludized on a microfludizer in the proper smaller chamber to the final particle size maximum of 500 nm with a pressure of 1000 -2000 Bar, at the temperature higher than 0° C. until the final particle size smaller than 500 nm with a pressure of 1000-2000 Bar, at the temperature higher than 0° C.; the resulting suspension is then mixed with a translucent pharmaceutical gel in ratios of 10:1 to 1:10.

Liposomal gel hydrophobic phthalocyanine preparation for photodynamic therapy of tumors and other diseases; The approach in therapeutic use is that the preparation is applied onto the tumor surface or the pathological part of the body and is let to act for the time period of one minute up to 24 hours, and afterwards, the location is irradiated by the light of the wavelength between 500 to 800 nm and intensity of at least 1 J/cm². The resulting gel containing liposomes with a curing drug is during therapy applied onto surface tumors in dermatology or other tumors accessible for light-delivering endoscopes and ideally after several minutes is irradiated by light of the desired wavelength. The suggested system enables instant penetration of the curing drug into the tumor and nearly immediate irradiation (in a minute time intervals from the application) with disintegration curing effect on the tumor. Such a high disintegration effect of the suggested therapy is determined by the suggested composition of the gel.

### EXAMPLES OF TECHNOLOGY

### Examples of Microfludization Procedure

### Example #1

On a microfluidizer, i.e. an instrument from Microfluidics, Inc., USA, of a laboratory or industrial type, there is at first microfludized a powder lecithin of a pharmacological purity at concentration between 10 to 30 mg per ml of sterile isotonic solution. Microfludization is conducted e.g. in the Z-chamber of 100 micrometer in diameter by several cycles so that the whole volume of the fluid is by several times cycled across the microfludization chamber, at a pressure of at least 1000 Bar. Afterwards, while constantly stirring the curing powder or the treated curing powder FCH is added according to the points 2 to 5 at ratios between 2:1 in relation to the lecithin. Subsequently, the suspension is again microfludized in the Z-chamber of 100 micrometer in diameter and at least by 100 cycles passages of the whole volume of fluid at the pressure of more than 1500 Bar and slush cooling. The fourth step is microfludization in a Z-chamber of 50 micrometer in diameter by at least 100 cycle passages of the fluid at the at the pressure of more than 1500 Bar and slush cooling.

### Example #2

On a microfluidizer, i.e. an instrument from Microfluidics, Inc., USA, of a laboratory or industrial type, there is at first microfludized the powder lecithin of a pharmacological purity at concentration between 10 to 30 mg per ml of sterile isotonic solution. Microfludization is conducted in the Z-chamber of 100 micrometer in diameter by several cycles so that the whole volume of fluid is by several times cycled across the microfludization chamber, at a pressure of more than 1000 Bar. Afterwards, in parallel the curing powder or the treated curing powder FCH is microfludized (usually in ratio to 2:1 related to the lecithin in an equal volume of isotonic solution), on a microfludizer in the Y-chamber of 100 or 75 micrometer in diameter by at least 100 cycle passages of the fluid at a pressure of more than 1500 Bar and slush cooling. Afterwards both microfludized components are mixed together in the Z-chamber of 100 micrometer in diameter by at least 100 cycle passages of the fluid at a pressure of more than 1500 Bar and slush cooling. The last step is again microfludization on a microfludizer in the Z-chamber of 50 micrometer in diameter by at least 100 cycle passages of the fluid at a pressure of more than 1500 Bar and slush cooling.

### Example #3

On a microfluidizer, i.e. an instrument from Microfluidics, Inc., USA, of a laboratory or industrial type, there is at first microfludized the powder lecithin of a pharmacological purity at concentration between 10 to 30 mg per ml of sterile isotonic solution. Microfludization is conducted e.g. in the Z-chamber of 100 micrometer in diameter by several cycles so that the whole volume of fluid is by several times cycled across the microfludization chamber, at a pressure of more than 1000 Bar. Afterwards, the curing powder or the treated curing powder FCH is added in the ratio of 2 to 1 related to the lecithin. Then the suspension is microfludized on a microfludizer in the Y-chamber of 100 or 75 micrometer in diameter by at least 100 cycle passages of the whole volume of fluid and a pressure of more than 1500 Bar and slush cooling. Then follows microfludization on a microfludizer in the Z-chamber of 100 micrometer in diameter by at least 100 cycle passages of the fluid at a pressure of more than 1500 Bar and slush cooling. During the last step, the suspension is again microfludized on a microfludizer in the Z-chamber of 50 micrometer in diameter by at least 100 cycle passages of the fluid at a pressure of more than 1500 Bar and slush cooling.

### Example #4

Lecithin or other lipid in the pharmacological purity at the concentration of 10 to 30 mg per milliliter of sterile isotonic solution is after solubilization treated by extrusion across the filters with sizes 100 to 500 nm. The resulting liposomes are then mixed with the curing drug or the treated curing drug according to the points 2 to 5 in the ratio of 5:1 to 0.1:1 related to the lecithin (lipid) and is again treated by extrusion across the filters with sizes 100 to 500 nm. In the end, the resulting suspension may be treated as described in the above Examples #1 to #3.

### Examples of Treatment of Amorphous or Powder Curing Drug

### Example #5

Fine powder FCH is coated by glucose in a ratio of 5-10% per one gram of FCH.

### Example #6

Fine powder FCH is coated by polyethyleneglycole PEG600 in a ratio of 5-10% per one gram of FCH.

### Example #7

Fine powder FCH is coated by lecithin of a pharmacological purity in a ratio of 5-10% per one gram of FCH.

### Example #8

Fine powder FCH is coated by sodium chloride (NaCl) in a ratio of 5-10% per one gram of FCH.

### Example #9

Fine powder FCH is hydrated onto an aqueous paste containing 25% of dry FCH.

### Examples of Mixing the Gel with Resulting Liposomes

### Example #10

4% carboxymethylcellulose in sterile water

Preservative: 4% Parabenum

Sterile water added up to 100 ml

### Example #11

4% carboxymethylcellulose in sterile water

Preservative: 2% Parabenum

Sterile water added up to 100 ml

### INDUSTRIAL APPLICABILITY

Liposomal gel hydrophobic phthalocyanine preparation is usable in medicine according this invention for therapy of tumors and other diseases.

1. Liposomal gel hydrophobic phthalocyanine preparation for photodynamic therapy of tumors, characterized in that it based on the system of lecithin liposomes or liposomes on the basis of other lipids, with incorporated curing drug, a photosenzitizer, which can be either one from a group including hydrophobic hydroxyaluminum phthalocyanine, hydrophobic aluminum phthalocyanine, hydrophobic zinc phthalocyanine, hydrophobic silicone phthalocyanine or organic silicone phthalocyanine, or hydrophobic phthalocyanine without the core metal; while resulting liposomes are mixed in ratios of 10:1 to 1:10 with a translucent gel, advantageously on the basis of carboxymethylcellulose.

2. Liposomal gel hydrophobic phthalocyanine preparation according to claim 1, characterized in that it is prepared from particles of the added curing drug coated by glucose or other saccharide in a ratio of 5-10% per gram of FCH.

3. Liposomal gel hydrophobic phthalocyanine preparation according to claim 1, characterized in that it is prepared from particles of the added curing drug coated by polyethyleneglycol or other usable polymer in a ratio of 5-10% per gram of FCH.

4. Liposomal gel hydrophobic phthalocyanine preparation according to claim 1, characterized in that it is prepared from particles of the added curing drug coated by lecithin or other lipid of the pharmaceutical purity in a ratio of 5-10% per gram of FCH.

5. Liposomal gel hydrophobic phthalocyanine preparation according to claim 1, characterized in that it is prepared from particles of the added curing drug coated by NaCl or other salt usable in pharmacology in a ratio of 5-10% per gram of FCH.

6. Method of the preparation of the liposomal gel hydrophobic preparation characterized in that lethicin or other lipids of the pharmaceutical purity in concentration from 1 to 40 mg per ml of fluid, advantageously of sterile isotonic solution, fluidizing on a microfluidizer in particular chamber into final particle size smaller than 1000 nanometer in temperature higher than 0° C. and with pressure 500 to 2000 Bar, then with constant stirring is added the curing drug or the treated curing drug according to the points 2 to 5 is added in ratios 5:1 to 0.1:1 in relation to lethicin or other lipid, the resulting suspension is again fluidized on a microfluidizer in particular smaller chamber into final particle size smaller than 500 nanometer with pressure 1000 to 2000 Bar in temperature higher than 0° C., the resulting suspension is then mingled with translucent gel in ratio between 10:1 to 1:10.

7. Method of manufacturing procedure for the liposomal gel hydrophobic phthalocyanine (FCH) characterized in that lecithin or other lipid of a pharmacological purity at concentration between 10 to 40 mg per ml of sterile isotonic solution is fluidized in microfluidizer in particular chamber into final particle size smaller than 1000 nanometer with pressure at least 1000 to 2000 Bar in temperature higher than 0° C.; Afterwards, the curing drug or the treated curing drug is separately fluidised on a microfluidizer in a particular chamber according to the points 2 to 5 in amounts corresponding to ratio between 5:1 to 0.1:1 in relation to lethicin or other lipid in the same volume of fluid, advantageously on sterile isotonic solution to the final particle size smaller than 1000 nanometer with pressure at least 1000 to 2000 Bar; Afterwards both microfludized components are mixed together and again microfludized on a microfludizer in particular chamber with pressure at least 1000 to 2000 Bar in temperature higher than 0° C. to the final particle size smaller than 500 nanometer; the resulting suspension is fluidised on a microfluidizer in a particular smaller chamber with pressure at least 1000 to 2000 Bar, in temperature higher than 0° C. to the final particle size smaller than 500 nanometer; the resulting suspension is then mixed with a translucent gel in ratios of 10:1 to 1:10.

8. Method of manufacturing procedure for the liposomal gel hydrophobic phthalocyanine (FCH) characterized in that lecithin or other lipid of a pharmacological purity at concentration between 10 to 40 mg per ml of sterile isotonic solution is treated by extrusion across the filters with sizes 10 to 500 nm together with the curing drug or the treated curing drug according to the points 2 to 5 in the ratio of 5:1 to 0.1:1 in relation to the lecithin or other lipid, the resulting suspension is again fludized on a microfludizer in particular chamber to the final particle size maximum of 500 nanometer, with pressure at least 1000 to 2000 Bar, in temperature higher than 0° C. to the final particle size smaller than 500 nanometer, with pressure at least 1000 to 2000 Bar, in temperature higher than 0° C.; the resulting suspension is then mixed with a translucent gel in ratios of 10:1 to 1:10.

9. Use of the liposomal gel phthalocyanine preparation for photodynamic therapy of tumors and other diseases.

10. Method of use of the liposomal gel phthalocyanine preparation for photodynamic therapy of tumors and other diseases characterized in that the preparation is applied onto the tumor surface or the pathological parts of the body, preparation is let to function for a period of time one minute up to 24 hours, and afterwards, the location is irradiated or illuminated by the light of the wavelength between 500 to 800 nm and intensity of at least 1 J/cm².
